(19) United States
(12) Patent Application Publication
(10) Pub. No.: US 2025/0262075 A1
(43) Pub. Date: Aug. 21, 2025

(54) PRELOADED DELIVERY SYSTEM FOR A PREFORMED STENT INTRODUCTION

(71) Applicant: COOK MEDICAL TECHNOLOGIES LLC, Bloomington, IN (US)

(72) Inventor: Urszula Salaj-Kosla, Limerick (IE)

(21) Appl. No.: 19/044,018

(22) Filed: Feb. 3, 2025

Related U.S. Application Data
(60) Provisional application No. 63/554,592, filed on Feb. 16, 2024.

(51) Int. Cl. A61F 2/966; A61M 25/00; A61M 25/01; A61M 25/06

(52) U.S. Cl. CPC A61F 2/966; A61F 2250/0098; A61M 25/002; A61M 25/0097; A61M 25/0102; A61M 25/0108; A61M 2025/0681; A61M 2205/0266

(57) ABSTRACT

A kit for storing a preformed stent includes a stent delivery system, a preformed stent and a flexible member. The stent delivery system includes a pushing catheter, and guiding catheter. The preformed stent has two configurations, a first configuration that has a non-linear shape and a second configuration that has a linear shape. The flexible member is also transformable from a first configuration that has a non-linear shape to a second configuration that has a linear shape. The flexible member and the preformed stent are in the first configuration when stored in the kit.

### INTRODUCTION

The present disclosure relates to a delivery system for an implantable medical device and more particularly to a method of storing the delivery system.

As is well known to those skilled in the art, the human body is composed of a plurality of interconnected systems that include organs, arteries, veins, ducts, and vessels. Some of the systems in the human body include the biliary system, respiratory system, the vascular system, and the renal system. The biliary system has the gall bladder and a plurality of bile ducts that are responsible for draining waste products from the liver into duodenum, which is part of small intestine, and to assist in digestion by releasing bile. The respiratory system includes a set of lungs that are covered by pleural membranes, a trachea that transports air from the nasal and oral cavities into the lungs. The respiratory system interacts with the vascular system through the pulmonary vein and pulmonary arteries that carry oxygenated blood from the lungs and deoxygenated blood from the heart to lungs, respectively. The vascular system includes the human heart and vessels, namely veins and arteries, that circulate blood all through the body.

Chronic ailments, physical trauma or a combination of both may directly or indirectly cause deterioration and malfunction of one or multiple body systems. An exemplary malady is, for instance, in the biliary system, an obstruction in the bile duct that affects the flow of bile from the liver to the duodenum. Similarly, performance of the vascular system is affected by presence of plaque in the arteries, and fluid buildup in the lungs, particularly in the pleural membrane, affects performance of the respiratory system.

Surgical interventions have been developed to alleviate and treat the aforementioned health issues. Implantable medical devices, such as stents, for example, the Cook Medical biliary stents and Cook Medical vascular stents, and drains, for example, the Cook Medical pleural drains and the Cook Medical nephroureterostomy drains, are devices that may be used in interventions to restore proper functionality of the bile, respiratory and renal body systems.

The design of the implantable medical devices used in surgical interventions may differ by shape, size, and material. These properties, ultimately, are dependent on the device's intended use, and the physiological anatomy of the intended deployment location in the human body.

The implantable devices are packaged and stored until needed for a surgical deployment procedure. The surgical practitioner is responsible for assembling the implantable medical device's delivery system and loading the implantable medical device onto the delivery system. Time needed for and complexity of assembling the delivery system and loading the implantable medical device on the delivery system is also dependent on the implantable medical device's shape, which may vary from a simple linear shape to a more complex nonlinear shape.

Thus, while current implantable medical devices and delivery systems achieve their intended purpose, there is a need for a new and improved system and method for storing and assembling a delivery system for an implantable medical device.

### SUMMARY

According to several aspects, a kit for deploying a preformed stent includes a stent delivery system, a preformed stent, and a flexible member. The stent delivery system includes a pushing catheter and a guiding catheter. The pushing catheter has an elongated tubular body. The elongated tubular body of the pushing catheter has a proximal portion, a distal portion, and a pushing catheter lumen extending between the proximal portion and the distal portion. The guiding catheter has an elongated tubular body. The elongated tubular body of the guiding catheter has a proximal portion, a distal portion, and a guiding catheter lumen extending between the proximal portion and the distal portion. The elongated tubular body of the guiding catheter is disposed in the pushing catheter lumen. The preformed stent has a tubular body. The tubular body has a proximal portion, a distal portion, and a stent lumen. The flexible member has an elongated cylindrical body. The elongated cylindrical body has a proximal portion and a distal portion. The proximal portion of the flexible member is disposed in the guiding catheter lumen. The distal portion of the flexible member is also disposed in the stent lumen.

In another aspect of the present disclosure, the pushing catheter includes a pushing catheter hub disposed at the proximal portion of the pushing catheter for receiving the guiding catheter.

In an additional aspect of the present disclosure, the guiding catheter includes a guiding catheter hub disposed at the proximal portion of the guiding catheter for receiving the flexible member.

In an additional aspect of the present disclosure, the guiding catheter includes a tapered end at the distal portion of the elongated tubular body of the guiding catheter to facilitate loading of the non-linear stent on the distal portion of the guiding catheter.

In an additional aspect of the present disclosure, the tubular body of the preformed stent has a first configuration that has a non-linear shape and a second configuration that has a linear shape.

In an additional aspect of the present disclosure, the first configuration of the tubular body of the preformed stent is a double pigtail shape.

In an additional aspect of the present disclosure, the flexible member includes a first configuration that has a non-linear shape and a second configuration that has a linear shape.

In an additional aspect of the present disclosure, the flexible member is a stylet.

In an additional aspect of the present disclosure, wherein the wherein the flexible member is made of nitinol.

In an additional aspect of the present disclosure, the preformed stent includes at least one radio opaque marker.

In an additional aspect of the present disclosure, wherein the guiding catheter has at least one radio opaque marker.

According to several aspects, a method of assembling a kit includes providing a pushing catheter that has an elongated tubular body that includes a proximal portion, a distal portion, and a pushing catheter lumen extending between the proximal portion and the distal portion. Additionally, the method includes disposing the guiding catheter in the pushing catheter lumen at the proximal portion of the elongated tubular body of the pushing catheter. The guiding catheter has an elongated tubular body that includes a proximal portion, a distal portion, and a guiding catheter lumen extending between the proximal portion and the distal portion. Furthermore, the method includes disposing a flexible stylet in the guiding catheter lumen at the proximal portion of the elongated tubular body of the guiding catheter. The flexible stylet has an elongated cylindrical body with a proximal portion and a distal portion. The method also includes providing a preformed stent that has a tubular body. The tubular body has a proximal end, a distal end and stent lumen. The tubular body also has a first configuration that has a non-linear shape and a second configuration that has linear shape. Additionally, the method includes disposing the elongated cylindrical body of the flexible stylet in the stent lumen at the proximal portion of the tubular body of the preformed stent. Furthermore, the method includes storing the assembled kit with the preformed stent in the first configuration.

In another aspect of the present disclosure, disposing the guiding catheter in the pushing catheter includes disposing the elongated tubular body of the guiding catheter in the pushing catheter lumen.

In an additional aspect of the present disclosure, disposing the flexible stylet in the guiding catheter includes disposing the distal portion of the elongated cylindrical body of the flexible stylet in the guiding catheter lumen until the distal portion of the elongated cylindrical body of the flexible stylet exits through the distal portion of the guiding catheter.

In an additional aspect of the present disclosure, disposing the flexible stylet in the preformed stent includes disposing the distal portion of the flexible stylet in the stent lumen.

In an additional aspect of the present disclosure, disposing the flexible stylet in the preformed stent includes transforming the tubular body of the preformed stent, in the first configuration, to the second configuration.

In an additional aspect of the present disclosure, disposing the flexible in the tubular body of the preformed stent includes transforming the elongated cylindrical body of the flexible stylet to the first configuration of the preformed stent.

In an additional aspect of the present disclosure, storing the assembled kit includes placing the kit in a container having a tray.

In an additional aspect of the present disclosure, storing the kit further includes securing the pushing catheter, the guiding catheter, the flexible stylet and the preformed stent on the tray.

According to several aspects, a method of delivering a preformed stent includes providing a kit. The kit includes a pushing catheter that has an elongated tubular body. The elongated tubular body has a proximal portion, a distal portion, and a pushing catheter lumen extending between the proximal portion and the distal portion. The kit further includes a guiding catheter that has at least one radio opaque marker and an elongated tubular body. The elongated tubular body of the guiding catheter also has a proximal portion, a distal portion, and a guiding catheter lumen extending between the proximal portion and the distal portion. The distal portion of the guiding catheter is disposed in the pushing catheter lumen. The kit further includes a flexible stylet that has an elongated cylindrical body. The elongated cylindrical body of the flexible stylet has a proximal portion and a distal portion. The proximal portion of the cylindrical body of the flexible stylet is disposed in the guiding catheter lumen. The kit further includes a preformed stent that has at least one radio opaque marker and a tubular body. The tubular body has a proximal portion, a distal portion, a stent lumen, a first configuration that has a non-linear shape, and a second configuration that has a linear shape. The distal portion of the flexible stylet is disposed in the stent lumen. The method further includes loading the preformed stent on to the guiding catheter, removing the flexible stylet from the guiding catheter lumen, advancing the guiding catheter with the preformed stent loaded on the distal portion of the tubular body of the guiding catheter through an endoscope positioned in the body, locating the guiding catheter in the body using the at least one radio opaque marker on the guiding catheter, locating the preformed stent in the body using the at least one radio opaque marker on the preformed stent, and deploying the preformed stent in the lumen of the body by contacting the proximal portion of the preformed stent with the distal portion of the pushing catheter and retracting the guiding catheter from the lumen of the preformed stent and the lumen of the pushing catheter.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a schematic illustration of a preformed stent delivery system and a preformed stent disposed on a flexible member, in accordance with an aspect of the present disclosure;

FIG. 2 is a partial longitudinal cross-sectional view of a pushing catheter, a guiding catheter and a flexible member at the proximal end of the preformed stent delivery system, in accordance with an aspect of the present disclosure;

FIG. 3 is a partial cross-sectional view of a guiding catheter with a tapered distal end and a flexible member disposed in the lumen of the guiding catheter, in accordance with an aspect of the present disclosure;

FIG. 4 is a partial cross-sectional view of the nonlinear portion of a preformed stent with a flexible member conforming to the shape of the preformed stent disposed in the lumen of the preformed stent, in accordance with an aspect of the present disclosure;

FIG. 5A is a perspective view of a preformed stent and a flexible member conforming to the shape of the preformed stent in a first configuration, in accordance with an aspect of the present disclosure;

FIG. 5B is a perspective view a preformed stent and a flexible member conforming to the shape of the preformed stent in in a second configuration, in accordance with an aspect of the present disclosure; and

FIG. 6 is a perspective view a kit including a preformed stent delivery system, a flexible member and a preformed stent secured to a tray and disposed in a container, in accordance with an aspect of the present disclosure.

### DETAILED DESCRIPTION

The following description is merely exemplary in nature and is not intended to limit the present disclosure, application, or uses.

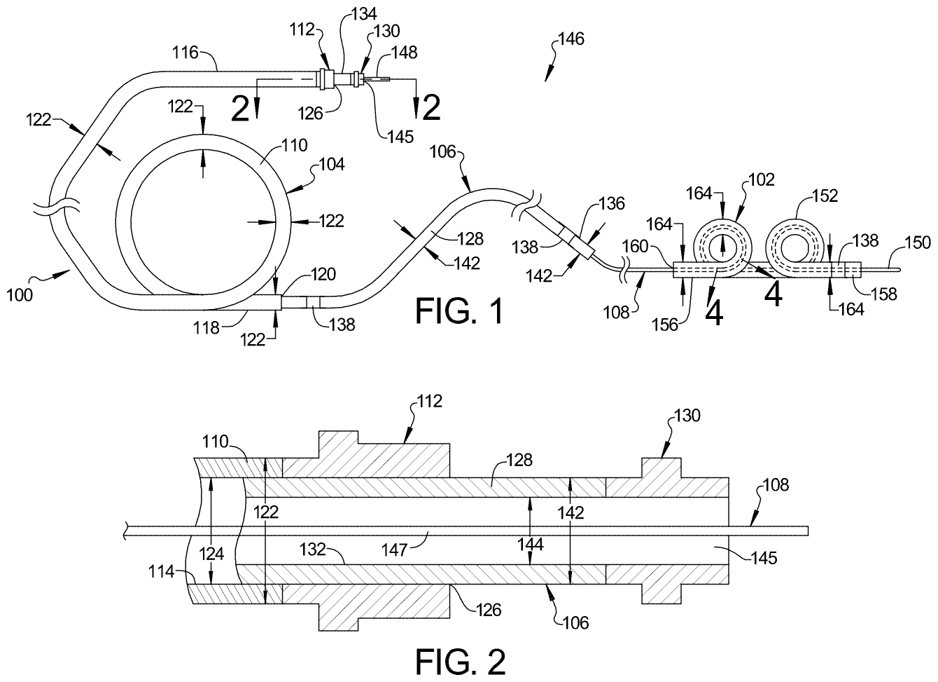

FIG. 1 is a perspective view illustrating a preformed stent delivery system 100, in accordance with the present disclosure. The preformed stent delivery system 100 is used for deploying a stent 102 in a body lumen (not shown). The stent delivery system 100 includes a pushing catheter 104 and a guiding catheter 106. The stent 102 for example is a preformed stent.

The pushing catheter 104 has an elongated tubular body 110 and a hub 112. The tubular body 110 defines a lumen 114 (shown in FIG. 2) and has a proximal end 116 and a distal end 118. The tubular body 110 also includes a contact surface 120 at the distal end 118 for contacting the preformed stent 102, as will be more fully described below. The length of the tubular body 110 of the pushing catheter 104 is for example 170 cm, 185 cm, 205 cm or as needed to perform the desired procedure. The tubular body 110 also has an outer diameter 122 of, for example, 8.5 Fr to 11.5 Fr, where 1 Fr is ⅓ mm, or as needed to perform the desired procedure. Furthermore, the lumen 114 defined by the tubular body 110 has an inner diameter 124 (shown in FIG. 2). The hub 112 is affixed to the tubular body 110 of the pushing catheter 104 at the proximal end 116. The hub 112 has an entrance port 126 to provide access to the lumen 114, and a hemostatic valve (not shown) closing the lumen 114 to prevent loss of bodily fluids when in use. The dimensions given in this paragraph are exemplary only and not intended in any way to be limiting.

The guiding catheter 106 has a tubular body 128 and a hub 130. The tubular body 128 defines a lumen 132 (shown in FIG. 2) and has a proximal end 134 and a distal end 136. Moreover, the tubular body 128 includes one or more radio opaque markers 138. The radio opaque markers 138 are disposed or affixed on the on the external surface on the tubular body 128, between the proximal end 134 and the distal end 136. The length of the guiding catheter 106, for example, is 320 cm or as needed to perform the desired procedure. The tubular body 128 has an outer diameter 142 that is sized such that the guiding catheter 106 can be disposed in the lumen 114 of the pushing catheter 104 through the entrance port 126 in the hub 112 affixed to the tubular body 110 of the pushing catheter 104. For instance, guiding catheter 106 with an outer diameter of 5 Fr is compatible with the lumen 114 of pushing catheter 104 with an outer diameter 122 of 8.5 Fr. The guiding catheter lumen 132 also has an inner diameter 144 (shown in FIG. 2) sized as needed to perform the desired procedure. The radio opaque markers 138 are spaced apart along the tubular body 128 of guiding catheter 106, for example, of 5 cm apart between the proximal end 134 and the distal end 136. Furthermore, the thickness of the radio opaque markers 138 disposed on the tubular body 128 of the guiding catheter 106 are sized such that the radio opaque markers 138 do not affect or inhibit the performance of guiding catheter 106. The hub 130 is affixed to the tubular body 128 of guiding catheter 106 at the proximal end 134. The hub 130 has an entrance port 145 and a locking mechanism (not shown). The entrance port 145 provides access to the lumen 132 and a hemostatic valve (not shown) closing the lumen 132 of guiding catheter 106 to prevent loss of bodily fluids when in use. The dimensions given in this paragraph are exemplary only and not intended in any way to be limiting.

A flexible member or stylet 108 is packaged with the stent delivery system 100 and the stent 102 to form a kit 146, as will be described in further detail below. The kit 146 is an assembly including the stent delivery system 100, the stylet 108 and the stent 102. The stylet 108 has an elongated, cylindrical body 147 with a proximal end 148 and a distal end 150. In an aspect of the present disclosure, the elongated cylindrical body 147 (shown in FIG. 2) of the stylet 108 is made of a metal compound, such as nitinol or similar material. In another aspect of the present disclosure, the cylindrical body 147 of the stylet 108 is made of a plastic, such as Nylon or similar material. Accordingly, the stylet 108 is configured to be more flexible than the guiding catheter 106. Furthermore, the selection of certain materials such as Nitinol allows the stylet 108 to take on a particular shape such as a single bend or multiple bends and keep that shape over extended periods of time. Moreover, the length of the elongated cylindrical body 147 is such that the cylindrical body 147 extends completely through the tubular body 128 of the guiding catheter 106. The thickness of the cylindrical body 147 of the stylet 108 is less than the inner diameter 144 of the lumen 132 of the guiding catheter 106 such that the stylet 108 is easily passed through the lumen 132 of the guiding catheter 106. Additionally, the distal end 150 has a shape, for example rounded, that prevents puncturing of the lumen 132 of the guiding catheter 106 during advancement of the stylet 108 through lumen 132. Furthermore, the proximal end 148 of the stylet 108 is configured to be locked in place with the locking mechanism on the hub 130 of the guiding catheter 106.

The preformed stent 102 has a tubular body 152 that defines a lumen 154 (shown in FIG. 4). The tubular body 152 includes a proximal end 156, a distal end 158, and at least one radio opaque marker 138. Furthermore, the tubular body has an outer diameter 164 that is sized as needed to perform the desired procedure. In a first configuration the tubular body 152 of preformed stent 102 has a nonlinear shape, for example, a double-pigtail shape. The tubular body 152 of the preformed stent 102 is also transformable into a second configuration. In the second configuration, for example, the tubular body 152 of the preformed stent 102 takes on a linear shape. The second configuration is achieved through the application of a tensile load on the tubular body 152 of the performed stent 102. Furthermore, the preformed stent 102 has a contact surface 160 on the proximal end 156 of the tubular body 152.

Referring again to FIG. 2, a longitudinal cross-sectional view of a portion of the pushing catheter 104 and guiding catheter 106 is illustrated, in accordance with the present disclosure. The tubular body 110 has outer diameter 122, and the lumen 114 has an inner diameter 124. Further illustrated is the tubular body 128 of the guiding catheter 106 having an outer diameter 142, and the lumen 132 having an inner diameter 144. The tubular body 128 of the guiding catheter 106 is further illustrated positioned in the lumen 114 of the pushing catheter 104. Additionally, the stylet 108 is shown disposed through in the lumen 132 of the guiding catheter 106.

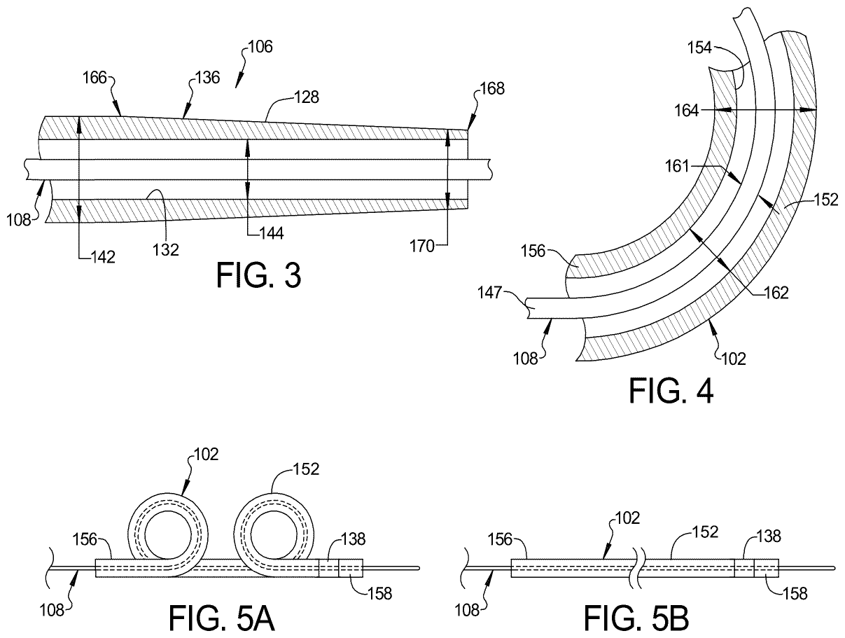

Referring now to FIG. 3, a cross-sectional view of the distal end of the guiding catheter 106 is illustrated, in accordance with the present disclosure. In an aspect of the present disclosure, the distal end 136 of the guiding catheter 106 is tapered from a first portion 166 remote from the distal end 136 of the guiding catheter 106 to a second portion 168 at the distal end 136. Accordingly, the outer diameter of the tubular body 128 changes from a first diameter 142 of the first portion 166 to a second diameter 170 at the second portion 168. Advantageously, the tapered distal end 136 of the guiding catheter 106 allows for easy loading of the preformed stent 102 on to the guiding catheter 106.

In still another aspect, the present disclosure contemplates that the guiding catheter 106 is configured to be compatible with both long wire and short wire guides (not shown). For example, as discussed in U.S. Pat. No. 7,967,830 (“the '830 patent”), the entire disclosure of which is incorporated herein by reference, the guiding catheter 106 includes an intra ductal exchange port (not shown) at the distal end 136 of the guiding catheter 106 for use with a short wire guide. The intra ductal exchange port provides access to the lumen 132 of the guiding catheter 106 at the distal end 136 to allow for insertion of the short wire guide as disclosed in the '830 patent. Moreover, the hub 130 of the guiding catheter 106 provides access to the lumen 132 through the entrance port 145 at the proximal end 134 of the guiding catheter 106, as described above, for insertion of a long wire guide.

Referring now to FIG. 4, a longitudinal cross-sectional view of a nonlinear portion of the preformed stent 102 and the lumen 154 is illustrated, in accordance with the present disclosure. As shown, the cylindrical body 147 of the stylet 108 has a diameter 161 that is less than an inner diameter 162 of the lumen 154 such that the cylindrical body 147 is easily passed through the lumen 154 of the preformed stent 102. Further illustrated is the stylet 108 conforming to the shape of the stent 102.

Referring now to FIGS. 5A and 5B, the two configurations of the preformed stent are illustrated, in accordance with the present disclosure. The stent 102 is shown in FIG. 5A in the first configuration that is non-linear and, for example, in a double pigtail shape. The second configuration shown in FIG. 5B is the linear or straight configuration or shape. The preformed stent is transformed from the first configuration (non-linear as shown in FIG. 5A) to the second configuration (straight or linear as shown in FIG. 5B) by locking or holding the proximal end 148 of the stylet 108 in place or stationary with the locking mechanism (not shown) on the hub 130 of the guiding catheter 106, and applying a tensile force on the distal end of the stylet 108. As illustrated in FIG. 5A, advantageously the stylet 108 conforms to the predetermined nonlinear shape, for example the double pigtailed shape, of the preformed stent 102.

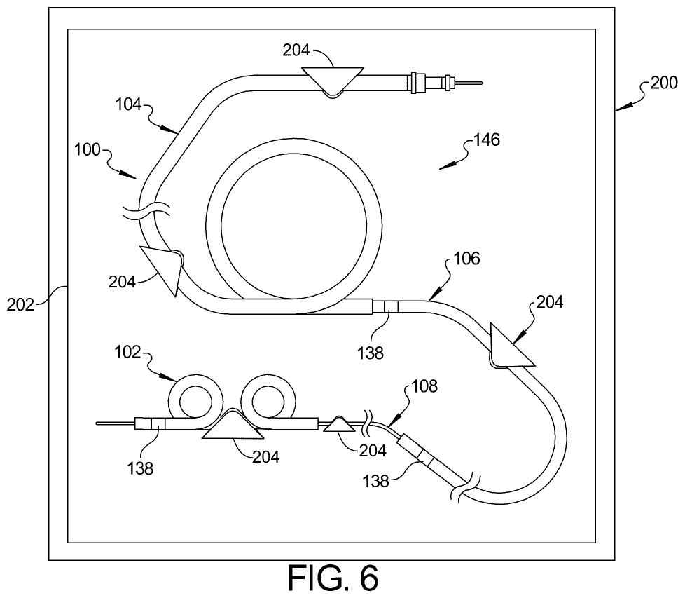

Referring now to FIG. 6, a perspective view of the kit 146 is illustrated disposed in a container 200, in accordance with present disclosure. The container 200 is a sealable enclosure that houses a tray 202. The tray 202 includes a plurality of fasteners 204. In an aspect of the present disclosure, the fasteners 204 are portions of the tray 202 that are cut out of the tray 202 and folded and placed over portions of the components in the kit 146, more particularly, portions of the stent delivery system 100, the stylet 108 and the stent 102 to secure same in place on the tray 202 during storage. The container 200 is configured to create a sterile and dry environment for the kit 146 to reside in during storage. The container 200 is also sized to stabilize and prevent movement of the tray 202 and the components of the kit 146 while in storage. In an aspect of the present disclosure, the container 200 is made from sterilized medical grade plastic that is abrasion resistant or similar materials used for packaging purposes. The tray 202 and the kit 146 are enclosed by the container 200, for example, that is sealed through direct sealing with a fibrous, porous top web, such as a flash-bonded high-density polyethylene (HDPE) or similar material for direct sealing.

The kit 146 is assembled with the preformed stent 102 in the first configuration, that is in the predetermined nonlinear shape of the preformed stent 102, as shown in FIG. 5A. In an exemplary aspect of the present disclosure, the kit 146 (shown in FIG. 1) is assembled by first disposing the guiding catheter 106 in the lumen 114 (shown in FIG. 2) of the pushing catheter 104 through entrance port 126 of the hub 112 affixed to the proximal end 116 of the pushing catheter 104. The guiding catheter 106 is advanced through the lumen 114 until the distal end 136 of the guiding catheter 106 exits tubular body 110 of the pushing catheter 104 at the distal end 118 of the pushing catheter 104. The stylet 108 is then disposed in the lumen 132 (shown in FIG. 2) of the guiding catheter 106 through entrance port 145 of the hub 130 until the distal end 150 of the stylet 108 exits the distal end 136 of the tubular body 128 of the guiding catheter 106. The distal end 150 of the stylet 108 is further advanced through the lumen 154 (shown in FIG. 4) of the preformed stent 102 until it exits the distal end 158 of the tubular body 152 of the preformed stent 102.

In another aspect, the kit 146 is assembled by creating a first subassembly. The first subassembly is formed by disposing the stylet 108 in the lumen 132 of the guiding catheter 106. The first subassembly including the stylet 108 and the guiding catheter 106 is then advanced through the lumen 114 of the pushing catheter 104 through the entrance port 126 in hub 112. The distal end 150 of the stylet 108 is further advanced through the lumen 154 of the preformed stent 102 until it exits the distal end 158 of the tubular body 152 of the preformed stent 102.

With continuing reference to FIG. 6, the kit 146 that includes the stent delivery system 100, stylet 108 and the preformed stent 102 is removed from the container 200 and detached from the tray 202, in preparation for a stent deployment procedure. The preformed stent 102 is then transformed from the first configuration to the second configuration. The transformation from the first configuration (non-linear, as shown in FIG. 5A) to the second configuration (linear, as shown in FIG. 5B) is achieved by locking or holding the proximal end 148 of the stylet in place or stationary, with the locking mechanism (not shown), for example, on the hub 130 of the guiding catheter 106, and then applying a tensile load to the distal end 150 of the stylet 108 while holding the proximal end 156 of the stylet 108. The preformed stent 102, now straightened and linear, is then loaded onto the distal end 136 of the guiding catheter 106 while the stylet 108 is still under the tensile load. Thus, loading of the stent 102 onto the guiding catheter 106 is performed more easily and quicker compared to other methods. After the stent 102 has been fully loaded and disposed onto the guiding catheter 106, the stylet 108 is removed from the stent delivery system 100 by retracting the proximal end 148 of the stylet 108 from the hub 130 at the proximal end 134 of the guiding catheter 106.

The stent delivery system 100 with the preformed stent 102 loaded on to the guiding catheter 106, and the stylet 108 removed, is then inserted into a body lumen or cavity to deliver the stent 102 to the intended destination in a patient's body. The locations of the guiding catheter 106 and the preformed stent 102 in the patient's body are monitored fluoroscopically by tracking the radio opaque markers 138 on the guiding catheter 106 and preformed stent 102.

When the preformed stent 102 has been advanced to the intended location in the body, the stent 102 is deployed by retracting the guiding catheter 106 and holding pushing catheter 104 positionally to prevent movement of the preformed stent 102. More specifically, when the guiding catheter 106 is retracted, the contact surface 120 at the distal end 118 of the pushing catheter 104 contacts the contact surface 160 at the proximal end 156 of the preformed stent 102 to prevent the preformed stent 102 from retracting with the guiding catheter 106.

In another aspect of the present disclosure, delivering the preformed stent 102 that is loaded on the guiding catheter 106 is performed with the assistance of either a long or short wire guide (not shown). When the preformed stent 102 has been advanced to the intended location in the body, the stent 102 is deployed by retracting the guiding catheter 106 and the wire guide, while holding the pushing catheter 104 positionally to prevent retraction of the preformed stent 102.

The stent delivery system 100 of the present disclosure offers several advantages. For example, loading the preformed stent 102 on the guiding catheter 106 with the aid of the stylet 108 simplifies the process by which the stent 102 is loaded onto the guiding catheter 106, and thus shortens the preparation time to prepare stent delivery system 100 and the preformed stent 102 for deployment procedure. Furthermore, the placement of preformed stent 102 on the stylet 108 while in storage preserves the nonlinear shape of the stent 102, and thus increases the shelf life of the kit 146.

The present disclosure contemplates assembling other aspects of a kit 146. Alternate kits may consist of only the preformed stent 102, the guiding catheter 106 and the stylet 108. In yet another aspect of the present disclosure, the preformed stent 102 is designed, sized, shaped and configured for use in, for example, percutaneous procedures, such as tracheostomy, cricothyrotomy, pleural drainage, and nephroureterostomy drainage. In still another aspect, the preformed stent 102 is designed, sized, shaped and configured for use in endoluminal procedures. In yet another aspect of the present disclosure, the preformed stent 102 has a nonlinear first configuration and a nonlinear second configuration.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A delivery system for deploying a preformed stent, the preformed stent having a tubular body, wherein the tubular body has a proximal portion, a distal portion, and a stent lumen, the system comprising:
a pushing catheter having an elongated tubular body, wherein the elongated tubular body has a proximal portion, a distal portion, and a pushing catheter lumen extending between the proximal portion and the distal portion of the elongated tubular body;
a guiding catheter having an elongated tubular body, wherein the elongated tubular body has a proximal portion, a distal portion, and a guiding catheter lumen extending between the proximal portion and the distal portion, and wherein the elongated tubular body of the guiding catheter is disposed in the pushing catheter lumen; and
a flexible member having an elongated cylindrical body, wherein the elongated cylindrical body has a proximal portion and a distal portion, wherein the proximal portion of the elongated cylindrical body of the flexible member is disposed in the guiding catheter lumen, and wherein the distal portion of the elongated cylindrical body of the flexible member is disposed in the stent lumen.

2. The delivery system of claim 1, wherein the pushing catheter further comprises a pushing catheter hub disposed at the proximal portion of the pushing catheter for receiving the guiding catheter.

3. The delivery system of claim 1, wherein the guiding catheter comprises a guiding catheter hub disposed at the proximal portion of the guiding catheter for receiving the flexible member.

4. The delivery system of claim 1, wherein the guiding catheter further comprises a tapered end at the distal portion of the elongated tubular body of the guiding catheter to facilitate loading of the tubular body of the preformed stent on the distal portion of the guiding catheter.

5. The delivery system of claim 1, wherein the tubular body of the preformed stent has a first configuration that has a non-linear shape and a second configuration that has a linear shape.

6. The delivery system of claim 5, wherein the first configuration of the tubular body of the preformed stent is a double pigtail shape.

7. The delivery system of claim 6, wherein the flexible member further comprises a first configuration that has a non-linear shape and a second configuration that has a linear shape.

8. The delivery system of claim 7, wherein the flexible member is a stylet.

9. The delivery system of claim 7, wherein the flexible member is made of nitinol.

10. The delivery system of claim 1, wherein the preformed stent further comprises at least one radio opaque marker.

11. The delivery system of claim 1, wherein the guiding catheter has at least one radio opaque marker.

12. A method of assembling a kit, the method comprising:
providing a pushing catheter having an elongated tubular body, wherein the elongated tubular body has a proximal portion, a distal portion, and a pushing catheter lumen, and wherein the pushing catheter lumen extends between the proximal portion and the distal portion of the elongated tubular body;
disposing a guiding catheter in the pushing catheter lumen at the proximal portion of the elongated tubular body of the pushing catheter, wherein the guiding catheter has an elongated tubular body having a proximal portion, a distal portion, and a guiding catheter lumen, and wherein the guiding catheter lumen extends between the proximal portion and the distal portion of the elongated tubular body of the guiding catheter;
disposing a flexible stylet in the guiding catheter lumen at the proximal portion of the elongated tubular body of the guiding catheter, and wherein the flexible stylet has an elongated cylindrical body having a proximal portion and a distal portion;
providing a preformed stent having a tubular body, wherein the tubular body has a proximal portion, a distal portion and a stent lumen, and wherein the tubular body has a first configuration that has a non-linear shape and a second configuration that has a linear shape;
disposing the distal portion of the elongated cylindrical body of the flexible stylet in the stent lumen of the tubular body of the preformed stent; and
storing the assembled kit with the preformed stent in the first configuration.

13. The method of claim 12, wherein disposing the guiding catheter in the pushing catheter further comprises disposing the elongated tubular body of the guiding catheter in the pushing catheter lumen.

14. The method of claim 12, wherein disposing the flexible stylet in the guiding catheter further comprises disposing the distal portion of the elongated cylindrical body of the flexible stylet in the guiding catheter lumen until the distal portion of the elongated cylindrical body of the flexible stylet exits through the distal portion of the guiding catheter.

15. The method of claim 14, wherein disposing the flexible stylet in the preformed stent further comprises disposing the distal portion of the elongated cylindrical body of the flexible stylet in the stent lumen.

16. The method of claim 15, wherein disposing the flexible stylet in the stent lumen further comprises transforming the tubular body of the preformed stent, in the first configuration, to the second configuration.

17. The method of claim 16, wherein disposing the flexible stylet in the stent lumen, further comprises transforming the elongated cylindrical body of the flexible stylet to the first configuration of the preformed stent.

18. The method of claim 12, wherein storing the assembled kit further comprises placing the kit in a container, and wherein the container includes a tray.

19. The method of claim 18, wherein storing the assembled kit further comprises securing the pushing catheter, the guiding catheter, the flexible stylet and the preformed stent on the tray.

20. A method of delivering a preformed stent in a lumen of a body, the preformed stent having a tubular body, wherein the tubular body has a proximal portion, a distal portion, and a stent lumen, the method comprising:
providing a kit, wherein the kit includes:
a pushing catheter having an elongated tubular body, wherein the elongated tubular body has a proximal portion, a distal portion, and a pushing catheter lumen extending between the proximal portion and the distal portion;
a guiding catheter having at least one radio opaque marker and an elongated tubular body, wherein the elongated tubular body has a proximal portion, a distal portion, and a guiding catheter lumen extending between the proximal portion and the distal portion, and wherein the distal portion of the guiding catheter is disposed in the pushing catheter lumen;
a flexible stylet having an elongated cylindrical body, wherein the elongated cylindrical body has a proximal portion and a distal portion, and wherein the proximal portion of the elongated cylindrical body of the flexible stylet is disposed in the guiding catheter lumen wherein the distal portion of the elongated cylindrical body of the flexible stylet is disposed in the stent lumen;
loading the preformed stent on to the distal portion of the tubular body of the guiding catheter;
removing the flexible stylet from the guiding catheter lumen;
advancing the guiding catheter with the preformed stent loaded on the distal portion of the tubular body of the guiding catheter through an endoscope positioned in the body;
locating the guiding catheter in the lumen of the body using the at least one radio opaque marker on the guiding catheter;
locating the preformed stent in the lumen of the body using the at least one radio opaque marker on the preformed stent; and
deploying the preformed stent in the lumen of the body by contacting the proximal portion of the preformed stent with the distal portion of the pushing catheter and retracting the guiding catheter from the lumen of the preformed stent and the lumen of the pushing catheter.
